(19) United States
(12) Patent Application Publication
(10) Pub. No.: US 2006/0020178 A1
(43) Pub. Date: Jan. 26, 2006

(54) System and method for assessing sleep quality

(75) Inventors: John G. Sotos, Palo Alto, CA (US); John L. Branscum JR., Belmont, CA (US)

Correspondence Address: APNEOS CORPORATION, 2033 RALSTON AVENUE #41, BELMONT, CA, 94002, US

(73) Assignee: Apneos Corp., Palo Alto, CA (US)

(21) Appl. No.: 11/230,942

(22) Filed: Sep. 19, 2005

Related U.S. Application Data
(63) Continuation-in-part of application No. 10/214,792, filed on Aug. 7, 2002.
(63) Continuation-in-part of application No. 10/721,115, filed on Nov. 24, 2003.
(63) Continuation-in-part of application No. 11/094,911, filed on Mar. 30, 2005.
(60) Provisional application No. 60/610,888, filed on Sep. 18, 2004.

(51) Int. Cl. A61B 5/00; A61B 5/08

(52) U.S. Cl. 600/300; 600/529

(57) ABSTRACT

A method for improved visualization of information related to the physiology of a sleeping patient is disclosed. Physiological information from the patient is obtained by a device, converted to digital format, and processed to yield epochs of time based on whether the patient was in a physiologically desirable or undesirable state. The duration of epochs is plotted as a series of rectangles and a quality-of-sleep score is calculated based on the epoch lengths.

### CROSS-REFERENCES TO RELATED APPLICATIONS

This application claims priority to U.S. Provisional Patent No. 60/610,888 filed Sep. 18, 2004, commonly assigned, and hereby incorporated by reference for all purposes.

This application is a continuation-in-part of application Ser. No. 10/214,792 filed Aug. 7, 2002 and commonly assigned.

This application is a continuation-in-part of application Ser. No. 10/721,115 filed Nov. 24, 2003 and commonly assigned.

This application is a continuation-in-part of application Ser. No. 11/094,911 filed Mar. 30, 2005 and commonly assigned.

### BACKGROUND OF THE INVENTION

The present invention generally relates to ways of characterizing health related disorders. More particularly, the invention provides a system and method for assessing quality of sleep in a mammal or other organism that sleeps. Merely by way of example, the invention is provides a numerical and/or graphical representation of certain aspects of an organism's sleep.

Many diseases and conditions are characterized by fatigue, tiredness, sleepiness, and/or related states. In some cases, inadequate sleep may contribute to such states. Thus, in trying to ascertain the cause of a patient's tiredness, sleepiness, fatigue, and/or related state, a physician or other health care provider will often want to have information about the adequacy of the patient's sleep.

Although there are distinctions between these states, many people, especially persons who are not health care professionals, use them in conversation without careful attention to the distinctions. Thus, in conversations with physicians or other health care providers,

Sleep may be inadequate in various ways. Merely by way of example, sleep may be inadequate in quantity or in quality.

In some cases the quantity of sleep may be ascertained by asking the patient how long he or she sleeps. Some patients cannot provide a reliable answer to this question. For this and other reasons, various techniques have been developed to provide information on how long a person sleeps. Merely by way of example, such techniques include polysomnography and actigraphy.

In some cases the quality of sleep may be ascertained by asking the patient how well he or she sleeps. Many people, however, find it more difficult to describe their sleep quality than their sleep quantity. Sleep quantity can, for example, be described in standard units of time such as hours, but sleep quality is often discussed in more vague terms such as “slept well,” “slept poorly,” and the like.

Various techniques have been tried in an effort to add rigor to assessments of sleep quality. Merely by way of example, these include questionnaires such as the Pittsburgh Sleep Quality Index. Questionnaires, however, are commonly regarded as less than fully objective because a subject may, consciously or unconsciously, alter responses in various ways, e.g. to minimize reported symptoms.

As an additional example, polysomnography has been used to measure sleep quality. Humans normally cycle through several “stages” of sleep during the night (e.g. stage 4 sleep, rapid eye movement sleep, etc.), and the duration of various stages of sleep can sometimes be related to sleep quality. However, polysomnography generally requires a skilled technician for successful use. Furthermore, due to the large number of sensors employed, polysomnography may disturb or otherwise affect the subject's sleep.

U.S. Pat. No. 6,468,234 (to van der Loos et al) teaches a method and apparatus for measuring sleep quality that utilizes sensors incorporated in a sheet that is laid on top of a conventional mattress on which the subject sleeps. Among the possible analyses of data from acquired by the aforementioned sensors is a count of body shift episodes during the night, from which an index of nightly restlessness (in units of events per hour) may be calculated.

U.S. Pat. No. 6,579,233 (to Hursh) teaches a relationship between sleep quality and sleep fragmentation. Chokroverty defines sleep fragmentation as ______ Awakenings from sleep are sometimes called “arousals.” Several types of environmental and physiological events may fragment (interrupt) sleep. Merely by way of example, sleep may be fragmented by arousals or awakenings. Arousals from sleep may occur abnormally frequently in some subjects. For example, sleep apnea and related conditions may be associated with frequent arousals from sleep.

U.S. Pat. Nos. 6,363,270 and 6,091,973 (to Colla and Brydon) teach a measurement of sleep quality based on correlating two or more physiological signals from a patient, then linearly or proportionally scaling the number of coincident changes in the correlated signals. The former disclosure further teaches that “a change in at least two sympathetic physiological variables . . . is indicative of an arousal.”

However, counting and linearly scaling the number of signal change events and/or arousals appears to have shortcomings. Under the so-called “sleep continuity” theory of restorative sleep, “the rate of periodic fragmentation of sleep and not simply [the] total number of arousals” is said to be a determinant of daytime performance and sleepiness (M. H. BONNET. Sleep. 1987; 10: 364-373).

Thus, U.S. Pat. Nos. 6,743,167 and 6,553,252 and 6,530,884 and 6,527,715 and 6,419,629 (to Balkin et al) teach that the effect of arousals is non-linearly dependent on inter-arousal duration. They say: “Available data suggest that five minutes is the approximate length of time required to return to recuperative sleep (stage 2 or deeper sleep) following an arousal to wake or stage 1 sleep. If many hours of sleep are obtained without interruption, then the delays make only a small difference in overall restoration of cognitive performance capacity. If sleep is interrupted with frequent awakenings, the delays in recuperation after each awakening will accumulate, and thus substantially reduce total cognitive performance capacity restored during the total sleep period.” Balkin et al teach how to estimate cognitive performance based upon a plurality of historical data be supplied to a model, but do not teach explicit assessment of sleep quality.

U.S. Pat. No. 6,045,514 (to Raviv and Weingarten) teaches a method of determining a breathing resistance index of a sleeping subject from a set of respiratory sounds.
Plots showing sleep time blackened through the night (Hida?)

From the above, it is desirable to have improved techniques for characterizing health related disorders, for example objective, practical, sleep quality assessments based on sleep fragmentation.

### BRIEF SUMMARY OF THE INVENTION

A method for improved visualization of information related to the physiology of a sleeping patient is disclosed. Physiological information from the patient is obtained by a device, converted to digital format, and processed to yield epochs of time based on whether the patient was in a physiologically desirable or undesirable state. The duration of epochs is plotted as a series of rectangles and a quality-of-sleep score is calculated based on the epoch lengths.

Various additional objects, features, and advantages of the present invention can be more fully appreciated with reference to the detailed description and accompanying drawings that follow.

### BRIEF DESCRIPTION OF THE DRAWINGS

FIG. 1 shows a data stream and periods of sleep and non-sleep according to an embodiment of the present invention.

FIG. 2 shows flowchart detail of the quality calculator, according to an embodiment of the present invention.

FIG. 3 shows representations of three restoration functions according to an embodiment of the present invention.

FIG. 4 shows a graphical representation of nine continuity times according to an embodiment of the present invention.

FIG. 5 shows an alternate graphical representation of nine continuity times and a graphical representation of quality index according to an embodiment of the present invention.

FIG. 6 shows a graphical representation of more than nine continuity times according to an embodiment of the present invention.

FIG. 7 shows a path of information, from subject to viewer, according to embodiment of the present invention.

### DESCRIPTION OF THE SPECIFIC EMBODIMENT

According to the present invention, techniques for characterizing health related disorders are provided. More particularly, the present invention provides improved methods and systems for assessing sleep quality. Merely by way of example, the invention provides an index associated with sleep quality and/or a graphical representation associated with sleep quality for a patient that sleeps.

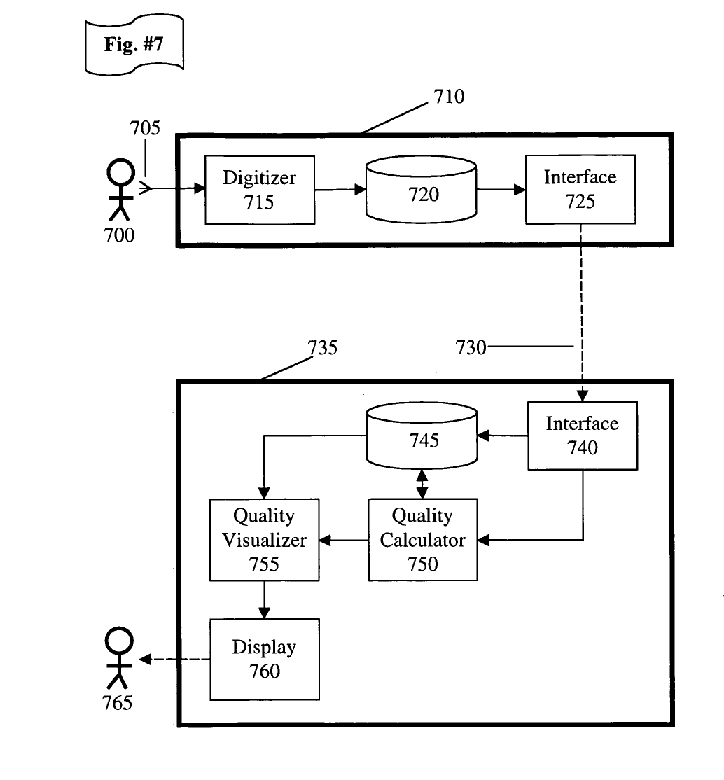

FIG. 7 shows an embodiment of the invention. A mammalian subject 700 sleeps (or attempts to sleep). During this time, one or more sensors 705 collect information from subject 700 and possibly from the subject's environment. This information is passed to a data collection device 710 having components 715, 720, and 725. If in analog form, information from sensors 705 is converted to digital form by a digitizing means 715, e.g. an analog-to-digital converter, to produce digital data. Digital data are stored in memory 720, which may be volatile or non-volatile.

The digital data are transferred to data analysis device 735. In an embodiment of the invention, the transfer need not occur at a specified time. Interface unit 725 in data collection device 710 passes digital data from memory 720 across a connection 730 to an interface unit 740 in data analysis device 735. Merely by way of example, connection 730 may be an electrically conductive cable or a wireless coupling. It may be analog or digital. Connection 730 may consist of multiple sub-connections, e.g. a communications network. In an embodiment of the invention, interface units 725 and 740 implement a USB (universal serial bus) connection. In another embodiment of the invention, interface units 725 and 740 implement a Bluetooth connection.

Interface 740 in data analysis device 735 may send digital data to quality calculator module 750 or store digital data in memory 745 for later retrieval by quality calculator module 750 or other components.

Quality calculator module 750 produces one or more indices of sleep quality based on the digital data. Merely by way of example, quality calculator module 750 may be implemented as a set of software codes. The sleep quality indices may be sent from quality calculator module 750 to quality visualization module 755, or be stored in memory 745 for later retrieval by quality visualization module 755 or other components.

Quality visualization module 755 produces information based on the indices of sleep quality that, when rendered by a display device 760, communicates information about the sleep quality of subject 700 to a human viewer 765. Merely by way of example, quality visualization module 750 may be implemented as a set of software codes. Viewer 765 may or may not be the same mammal as subject 700. In some cases viewer 765 may be a health care professional, e.g. a physician, veterinarian, psychologist, and the like.

Those with ordinary skill in the art will recognize that components of data collection device 710 and data analysis device 735 can be distributed among these or other devices in a plurality of ways. Merely by way of example, display 760 need not be part of data analysis device 735. Software codes may execute on different computers, e.g. software codes for quality calculator module 750 may execute on a computer that is networked to a computer upon which software codes for quality visualization module 755 execute.

In an embodiment of the invention, data analysis device 735 is a personal computer with appropriate software codes, and subject 705 is the same as viewer 765. This embodiment can yield convenient, private in-home assessment of sleep quality.

U.S. patent application Ser. No. 11/094,911 teaches an embodiment of data collection device 710. U.S. patent application Ser. Nos. 10/721,115 and 11/094,911 teach embodiments of sensor(s) 705. In an embodiment of the invention, data collection device 710 and data analysis device 735 are a single device.

The calculation and visualization of information about the sleep of subject 700 produced by quality calculator module 750 and quality visualization module 755 are based, in whole or in part, on the duration of sleep-related “continuity periods.” Continuity periods may be defined according to a plurality of criteria. An actigraphic criterion is outlined below.

In an embodiment of the invention, sensor(s) 705 include an actigraph. An actigraph senses acceleration of the body part to which it is mechanically coupled. Some actigraphs, for example, are worn like a wristwatch. In many cases, actigraphically-detected body motion during a period of sleep (or attempted sleep) is associated with wakefulness at or near the time of the motion. Similarly, periods of time in which an actigraph detects no acceleration may be associated with sleep. Actigraphs do not perfectly identify sleep and wakefulness, but some sleep physicians have found them useful nevertheless.

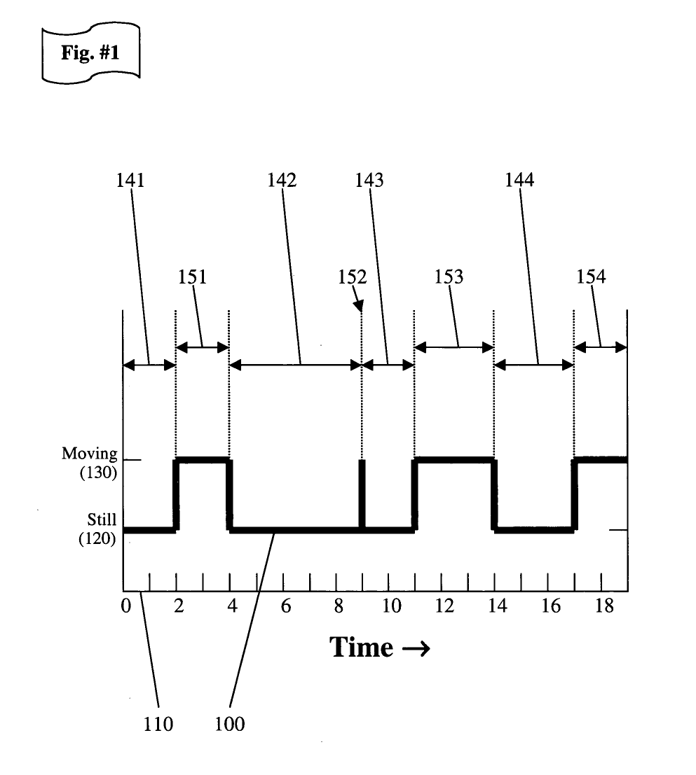

FIG. 1 shows a stylized actigraphic signal 100 over time. The signal is dichotomous over time, taking only the value “still” 120 (indicative of no actigraphic acceleration) or “moving” 130 (indicative of actigraphic acceleration). As an approximation, the value “still” 120 is associated with sleep, and “moving” 130 with wakefulness.

FIG. 1 may be conceptualized as periods of stillness interrupted by periods of movement (or, more interestingly but less accurately, as sleep interrupted by wakefulness). The continuously uninterrupted periods of stillness are “continuity periods.” Because signal 100 is based on actigraphy, we can say the continuity periods in FIG. 1 are defined according to an actigraphic criterion. Signal 100 may derive from other physiological data, including, but not restricted to, electroencephalographic (EEG) measurements, respiratory sound measurements, airflow measurements, arterial tonometry, combinations of these, and the like. Merely by way of example, in a case where an EEG criterion is used to define signal 100, signal values “still” 120 and “moving” 130 may be replaced with “asleep according to EEG” and “awake according to EEG,” respectively. Merely by way of example, when signal 100 is based on abnormalities of sleep breathing, signal values 120 and 130 may correspond to “no apnea/hypopnea” and “apnea or hypopnea,” respectively. In general, the values of signal 100 are associatable with desired-sleep and undesired-sleep-associated states (including wakefulness) of subject 700. For this reason, a period of time corresponding to an undesired-sleep-associated state is termed a “sleep interruption.” Continuity periods are the periods of time corresponding to desired-sleep-associated states.

Returning to FIG. 1, a continuity period 141 begins at time=0 and extends until time=2, when sleep interruption 151 begins. A second continuity period 142 begins at time=4 and extends until time=9, assuming that sleep interruption 152 is regarded as significant. (Various criteria may or may not regard all sleep interruptions as significant.) Other periods of sleep continuity (143, 144) and sleep interruption (153, 154) also appear in FIG. 1.

In an embodiment, continuity periods are characterized by a start, an end, and a duration of time.

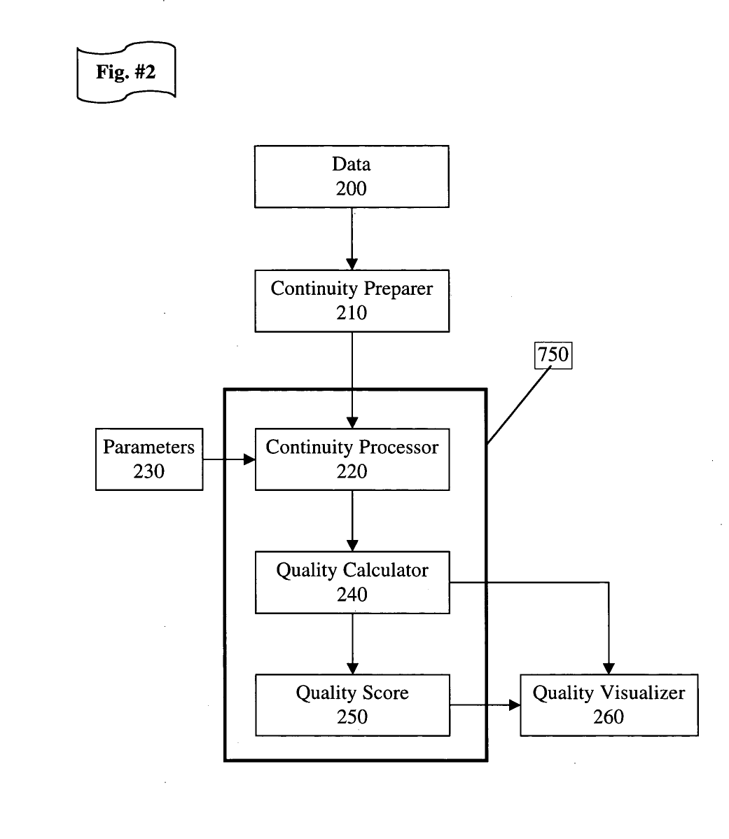

FIG. 2 shows detail of an embodiment of quality calculator 750. Digital data 200 correspond to a period of time associated with a sleep episode of a mammalian subject (e.g. as provided by interface 740). If data 200 are “raw,” optional continuity preparer 210 may reduce them to the form of signal 100. For example, measurements of actigraphic acceleration may be dichotomized into “still” and “moving” values according to some threshold.

Continuity processor 220 identifies zero or more continuity periods on the basis of data 200 and criteria parameters 230 for determining sleep interruption(s).

Each continuity period identified by continuity processor 220 has an associated duration in time, represented herein as durᵢ for continuity period i. In an embodiment, continuity processor 220 is implemented as software codes in a digital computing device. In an embodiment, continuity processor 220 calculates each durᵢ by subtracting the start-time of continuity period i from the end-time of continuity period i.

In an exemplary embodiment parameters 230 may include, but are not restricted to, the total duration of time represented by physiological data in data 200. Merely by way of example, parameters 230 may include, but are not restricted to, durₜₒₜ, the sum of durations of all continuity periods, where:

$${dur}_{tot} = {\sum }_{i} {dur}_{i} .$$

In an embodiment, continuity processor 220 includes a restoration function (possibly passed as a parameter 230) that maps a durᵢ to a dimensionless restoration value, restoreᵢ, ranging from 0 to 1 (inclusive) that represents the degree to which a period of uninterrupted sleep is restorative to the body and/or mind of a patient. A restoration value of 1, for example, indicates the continuity period was complete in its restorative effect while it occurred, whereas a restoration value of 0 would indicate the continuity period had no restorative effect. Higher restoration values normally contributoe to higher quality of sleep.

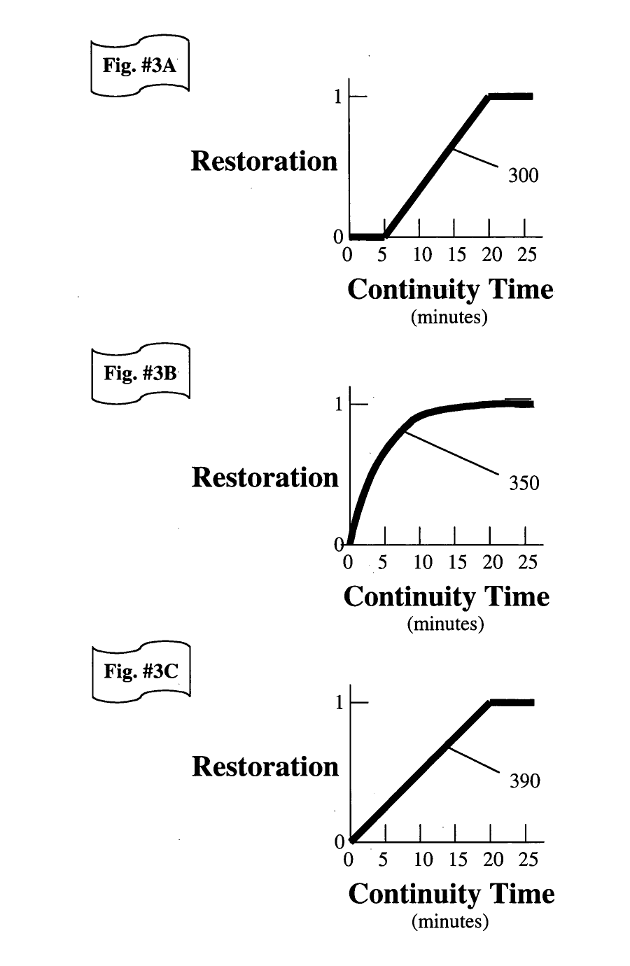

Merely by way of example, FIG. 3A graphically illustrates an exemplary restoration function 300; continuity time (durᵢ), in minutes, is plotted on the horizontal axis and restoration is plotted on the vertical axis. Restoration function 300 shows that continuity periods at most 5 minutes in length have no restorative effect, while continuity periods at least 20 minutes in length have a full restorative effect during their occurrence. Between 5 and 20 minutes, the relation between continuity time and restoration is linear and corresponds to a restoration range of 0 to 1; a continuity time of 10 minutes, for example, is associated with a restoration value of one-third.

FIG. 3B shows an alternative restoration function 350, derived from the work of Bonnet (Sleep. 1987; 10: 364-373). Restoration function 350 depicts a curvilinear, e.g. logarithmic, relation between continuity time and restoration.

FIG. 3B shows an alternative restoration function 390 wherein the relationship between continuity time and restoration is linear over continuity times of 0 to 20 minutes (corresponding to restoration values of 0 to 1), and flat thereafter.

Returning to FIG. 2, result(s) produced by continuity processor 220 are used to calculate 240 a quality score 250 and/or a visualization 260 of one or more aspects of quality. In an embodiment continuity processor 220 produces a (durᵢ, restoreᵢ) pair for each continuity period i. Quality calculator 240 uses these pairs and, optionally, elements of data 200 and/or parameters 230, to produce quality score 250. In an embodiment, quality calculator 240 is a set of software codes in a computer memory and data 200, parameters 230, and results from continuity processor 220 are stored in a computer memory.

In an exemplary embodiment quality score 250 is calculated 240 to be the sum of the restoreᵢ values over all continuity periods i. This embodiment, however, has the disadvantage that there is no pre-determined upper bound on the quality score, because the number of continuity periods might vary from patient to patient, or from sleep period to sleep period.

In an alternative exemplary embodiment, quality score 250 is calculated 240 to be the mean of the restoreᵢ values for all continuity periods i. This embodiment, however, has the disadvantage that multiple very short continuity periods, e.g. 30 seconds, would have an influence on the final quality score 250 out of proportion to their clinical importance. For example, under this embodiment, a first person who had 10 continuity periods of 30 seconds each at the beginning of an 8-hour night of sleep could potentially have a significantly different quality score 250 from a second person who had only one or two such periods at night's start, but otherwise slept identically to the first person the remaining 8 hours of the night. This difference in scores would be suspect, since many persons would claim that a few minutes at the beginning of a night's sleep is unlikely to greatly influence the restorative nature of the full night's sleep.

In an alternative exemplary embodiment, quality score 250 is calculated 240 as a higher statistical moment of the restoreᵢ values for all continuity periods i, e.g. the second statistical moment that is sometimes known as variance (to which the standard deviation is closely related), the third statistical moment that is sometimes known as skew, or the fourth statistical moment that is sometimes known as kurtosis, and the like. This embodiment has the same disadvantage as the embodiment using the first moment (mean) of the restoreᵢ values.

In an exemplary embodiment, calculation 240 of quality score 250 includes use of restoreᵢ values as weightings for durᵢ values over all continuity periods, e.g.:

$$quality score = {\sum }_{i} {restore}_{i} * {dur}_{i}$$

where * is the symbol for multiplication. In this embodiment, the quality score may be likened to total restorative sleep time. This embodiment has the advantage that multiple short-continuity periods should not, in most cases, change the quality score appreciably when there are long continuity period(s) also present.

In an embodiment, restoreᵢ values are used as weightings for durᵢ values and the sum over all continuity periods is normalized by durₜₒₜ, e.g.:

$$quality score = \frac{{\sum }_{i} {restore}_{i} * {dur}_{i}}{{dur}_{tot}} = “ quality index . ”$$

In this embodiment, the quality score above, hereinafter referred to as the “quality index,” is dimensionless and ranges from 0 to 1. This embodiment, too, is relatively insensitive to the presence of multiple short-continuity periods.

Although steps 210, 220, 230, and 240 are shown as distinct in FIG. 2, other arrangements are possible. Merely by way of example, feedback from one or more steps may be used as input to refine the operation of one or more other steps. As an additional example, a first continuity period may complete steps 210 and 220 before a second continuity period finishes step 210. Persons with ordinary skill in the art will recognize other possibilities.

Although providing a single number as a quality score for a patient's sleep episode(s) has advantages, there is a loss of information associated with reduction to a single number. Merely by way of example, clinicians may derive additional useful information from consideration of individual continuity periods. Such information may be provided with alphanumeric characters, possibly in tabular form. It may also be provided in graphical form by the quality visualizer 250.

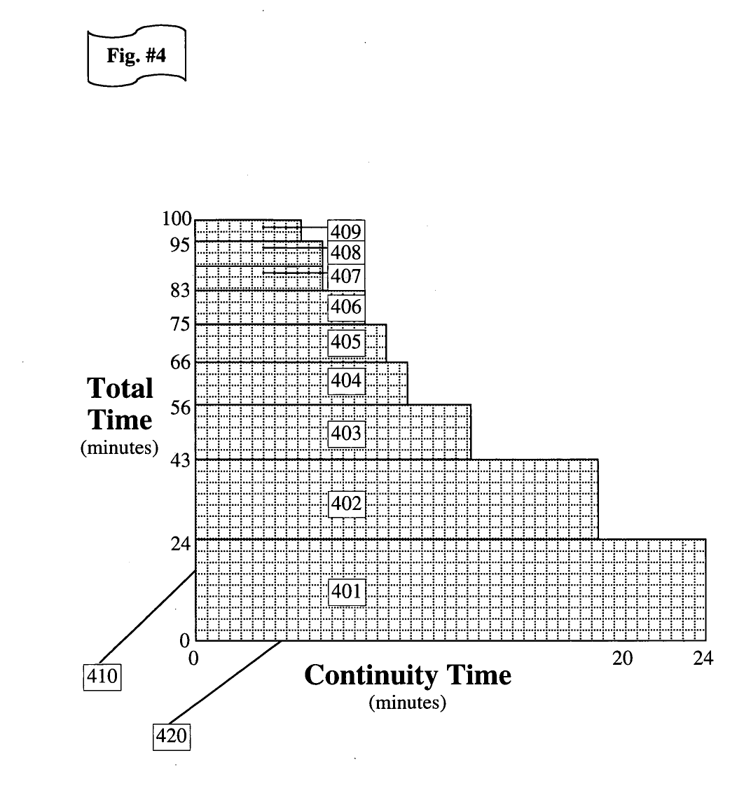

FIG. 4 shows an exemplary embodiment for visualizing a sleep period having 9 associated continuity times of 24, 19, 13, 10, 9, 8, 6, 6, and 5 minutes, respectively. In FIG. 4, each of the 9 continuity periods is drawn as a rectangle having interior hatch-lines (items 401-409, respectively). The rectangles are drawn left-justified to the vertical axis 410. The rectangles are drawn one atop the other, starting with the longest continuity period's rectangle 401 at the lowest extreme of the vertical axis 410 and proceeding to successively shorter continuity periods' rectangles 402-409. The continuity periods need not have occurred during the sleep period in the order in which they are drawn in FIG. 4. The durₜₒₜ is 100 minutes for the continuity periods in FIG. 4.

The height and width of each rectangle in FIG. 4 corresponds to the duration of the associated continuity period. The horizontal axis 420 and the vertical axis 410 have different scales, so, in general, each continuity period has the appearance of a non-square rectangle.

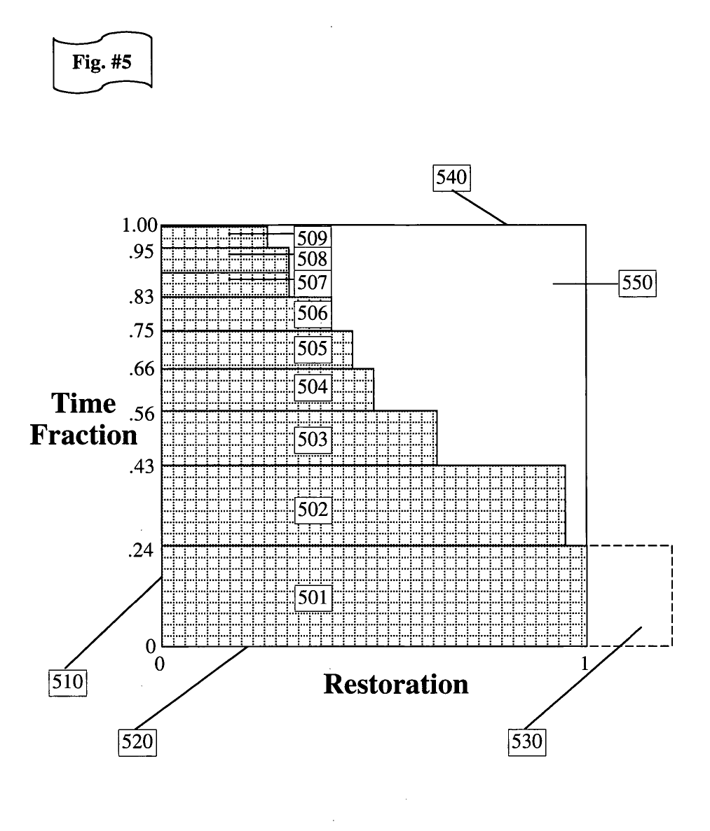

FIG. 5 shows another exemplary embodiment visualizing a sleep period having the same set of continuity durations (and durₜₒₜ) as FIG. 4. Compared to FIG. 4, each continuity period's rectangle (501-509) has the same proportional vertical extent, but values of the vertical axis 510 have been normalized with respect to durₜₒₜ. Vertical axis 510 is now labeled “Time Fraction.” In other words, the vertical extent of each rectangle 501-509 represents the proportion of durₜₒₜ attributable to the corresponding continuity period.

In FIG. 5 the horizontal extent of each continuity period's rectangle 501-509 corresponds to the restoreᵢ value for each continuity period, according to the restoration function of FIG. 3C. Because FIG. 3C specifies restoreᵢ=durᵢ/20 for durᵢ in minutes from 0 to 20, the relative horizontal extent of rectangles 502-509 in FIG. 5 does not change in comparison to rectangles 402-409 of FIG. 4. Rectangle 501 is, however, not as wide as rectangle 401, as shown by the dashed rectangular area 530 that was part of rectangle 401 but is not part of the rectangle 501. Rectangular area 530 is not part of rectangle 501 because the restoration function of FIG. 3C has a value of 1 at durᵢ≧20 minutes.

The similarity between the rectangles 401-409 and the rectangles 501-509 is advantageous. The concept of continuity time is likely to be more readily grasped by clinicians who first encounter the present invention than is the concept of restoration. The similarity in rectangles is expected to help introduce clinicians to the concept of restoration. Thus, in some cases where time fraction and restoration are plotted (viz. FIG. 5) it may be helpful to plot dashed rectangular areas (e.g. 530) to further reinforce the relationship between continuity time and restoration.

In FIG. 5 time fraction values on the vertical axis 510 and restoration values on the horizontal axis 520 both have limits from 0 to 1, inclusive. A frame 540 graphically indicates these limits. In an embodiment, vertical axis 510 and horizontal axis 520 are scaled to have an aspect ratio equal to 1 so that frame 540 is square.

A feature of the present invention is that the sum of the areas of rectangles 501-509 (as measured according to the scales of vertical axis 510 and horizontal axis 520) is equal to the quality index described above. A further feature of the present invention is that the cross-hatched area of FIG. 5, defined as the union of the extents of rectangles 501-509, represents the quality index when compared to the area enclosed by frame 540. Thus, the invention provides for a graphical means to communicate information about the duration and restoration of individual continuity periods as well as information about sleep quality for the period of sleep associated with the individual continuity periods.

Yet another feature of the present invention is that the limits on the axes 510 and 520 may be held constant regardless of patient or sleep episode. As a result, FIG. 5 may be simplified by removing various graphical elements to yield a simpler figure that practiced clinicians will still be able to interpret successfully. For example, numerical labels on axes 510 and 520 may be removed, rectangular area 530 may be removed, and borders common to more than one of rectangles 501-509 may be removed so that rectangles 501-509 are represented as a single polygon rather than stacked rectangles.

Various numerical annotations may be made as well. For example, the quality index may be printed over the cross-hatched area within frame 540, and 1 minus the quality index may be printed in the non-cross-hatched area 550 within frame 540.

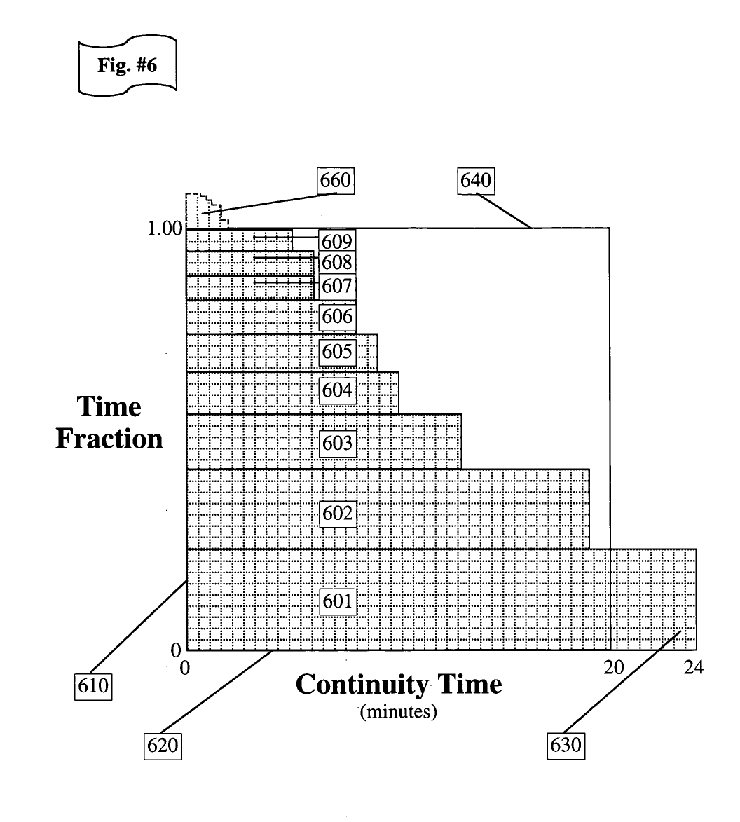

In some cases it may not be preferable to use restoration values to determine the horizontal extent of graphical representations of continuity periods, as was used for rectangles 501-509. Merely by way of example, there may be no restoration function of adequate reliability available for a certain subset of patients. FIG. 6 shows that, like FIG. 4, continuity time may be used to scale graphical representations of continuity periods along a horizontal axis 620 to produce rectangles 601-609. FIG. 6, however, adds frame 640 that serves a similar purpose to frame 540, i.e. it suggests upper bounds on values along vertical axis 610 and horizontal axis 620. In the case of FIG. 6, however, the suggested upper bound of horizontal axis values is 20 minutes. Thus, although the full horizontal extent of rectangle 601 is plotted, frame 640 graphically suggests that portion 630 (beyond 20 minutes) of rectangle 601 is of lesser importance.

Frame 640 in FIG. 6 can also be used to suggest lower bounds on the duration of continuity periods. In addition to the continuity periods used in FIG. 4 and FIG. 5, FIG. 6 represents several continuity periods of 2-or-fewer minutes using polygon 660. Polygon 660 is composed of stacked rectangles whose common borders have been hidden, each rectangle corresponding to a continuity period. Because rectangle 609 is within frame 640, someone viewing FIG. 6 might (correctly) conclude that frame 640 shows that the minimum duration of continuity times of greater importance is somewhere between 5 minutes (the width of rectangle 609) and 2 minutes (the maximum width of polygon 660).

It is seen in FIG. 6 that time fraction values (and, therefore, the vertical extent of rectangles 601-609) have been normalized to the total duration of continuity periods lying, in whole or in part, within frame 640. Thus, the definition of durₜₒₜ is different in FIG. 5 and in FIG. 6. In FIG. 6:

$${dur}_{tot} = {\sum }_{i} \{ \begin{matrix} 0 , & if {dur}_{i} \leq threshold \\ {dur}_{i} , & if {dur}_{i} > threshold\end{matrix}$$

where threshold is a duration in minutes (FIG. 6 used the example of 2 minutes), durᵢ is the duration of continuity period i, and the summation is over all continuity periods i.

In some embodiments, it may be advantageous to define durₜₒₜ as the subject's time-in-bed.

In an embodiment, different types of continuity periods may be defined and visualized according to the invention. For example, signal 100 may not be dichotomously valued, and a continuity period's type may vary according to the value of signal 100 ending the period. As a specific example, in a variant of signal 100, sleep may have value 0, an apnea event may have value 1, a hypopnea event may have value 2, an oxygen desaturation may have value 3, and so on. Types of continuity periods would correspond to periods ended by an apnea, hypopnea, oxygen desaturation, etc. Variations of shape, color, or other graphical features may be incorporated into visualization of such continuity periods, depending on their type.

In an embodiment, the duration of time between continuity events may also be figured into visualization or calculation of indices.

Other variations of quality calculator 240 and quality visualizer 260 are possible. In the boundary value case where there are zero continuity periods to be considered, a specific embodiment of quality calculator 240 would assign a reasonable value for quality score 240, e.g. zero.

The present invention has several potential advantages. Merely by way of example, perspicacious selection of restoration function and quality score definition can provide results in accord with the sleep continuity theory without undue influence from short periods of continuous sleep of lesser clinical importance. A further potential advantage of the present invention is the concise graphical representation of information about specific continuity periods that also communicates information about sleep continuity in aggregate.

Another potential advantage of the present invention is the ability to tailor sleep quality scores to various types of data 200. For example, a “sleep tranquility index” may be defined by using actigraphy data to determine when a patient moves during sleep. In a specific embodiment, periods without movement, i.e. tranquil periods, are defined as continuity periods. Thus, a higher value of the sleep tranquility index is associated with more tranquil sleep. In an alternative specific embodiment, any of several criteria may be used to determine when sleep has been interrupted. Merely by way of example, an interruption in sleep may be defined to occur when any of the following occur: an apnea, a hypopnea, a body movement, an oxygen desaturation, an electroencephalographic arousal, or a significant respiratory arrhythmia.

Another potential advantage of the present invention is its ability to facilitate comparisons between various criteria for interrupted and continuous sleep. Merely by way of example, different types of physiological events may predominantly occur in sleep apnea as compared to the upper airway resistance syndrome (UARS). Sleep apnea is generally characterized by repetitive apneas and hypopneas, whereas UARS is generally characterized by frequent respiratory effort-related arousals (RERAs). Continuity finder 210 could, in a first application of the present invention, be configured to include apneas and hypopneas as events indicating an interruption in sleep. Continuity finder 210 could then, in a second application of the present invention to data collected in association with the same sleep episode as the first application of the invention, be configured to additionally include RERAs as events indicating an interruption in sleep. The quality score 250 thereby produced and/or a representation thereby produced by quality visualizer 260 in each application of the invention could be compared to help understand the effect of different criteria in defining sleep interruption.

It should be noted that the above sequence of steps is merely illustrative. The steps can be performed using computer software or hardware or a combination of hardware and software. Any of the above steps can also be separated or be combined, depending upon the embodiment. In some cases, the steps can also be changed in order without limiting the scope of the invention claimed herein. One of ordinary skill in the art would recognize many other variations, modifications, and alternatives. It is also understood that the examples and embodiments described herein are for illustrative purposes only and that various modifications or changes in light thereof will be suggested to persons skilled in the art and are to be included within the spirit and purview of this application and scope of the appended claims.

What is claimed is:

1. A method for characterizing a state of a patient, comprising:
identifying a desired physiological state;
identifying an undesired physiological state;
obtaining physiological data from the patient;
identifying epochs of time wherein the physiological data are indicative of the subject having been in the desired physiological state without being in the undesirable physiological state;
rendering a graph wherein:
a first axis corresponds to length of time;
a second axis corresponds to fraction of the summed duration of all epochs;
each epoch is plotted as a rectangle according to the axes.
